SEQUENCE LISTING (20 sequences)

SEQ ID NO: 1 - DNA, 22 bp, rat
  misc_feature (1)..(22): sense primer
catctccttcccgccactcatc

SEQ ID NO: 2 - DNA, 21 bp, rat
  misc_feature (1)..(21): antisense primer
atacgcacgtagaccaggatc

SEQ ID NO: 3 - DNA, 20 bp, rat
  misc_feature (1)..(20): sense primer
ctggcmkrgcngacntcctg

SEQ ID NO: 4 - DNA, 21 bp, rat
  misc_feature (1)..(21): antisense primer
akrgyrtagatnanngggttc

SEQ ID NO: 5 - DNA, 20 bp, Artificial Sequence
  Description of Artificial Sequence sense primer sequence
cttcagcctccttgctatcg

SEQ ID NO: 6 - DNA, 20 bp, Artificial Sequence
  Description of Artificial Sequence antisense primer sequence
gcaggcaatgaagacactca

SEQ ID NO: 7 - DNA, 21 bp, Artificial Sequence
  Description of Artificial Sequence sense primer sequence
ccaaactacagcactctcatg

SEQ ID NO: 8 - DNA, 21 bp, Artificial Sequence
  Description of Artificial Sequence antisense primer sequence
gcttccttctaaaccacagag

SEQ ID NO: 9 - DNA, 20 bp, Artificial Sequence
  Description of Artificial Sequence sense primer sequence
tcagggagggcagtatgttc

SEQ ID NO: 10 - DNA, 20 bp, Artificial Sequence
  Description of Artificial Sequence antisense primer sequence
ctgactttcgaagaggatgg

SEQ ID NO: 11 - DNA, 20 bp, Artificial Sequence
  Description of Artificial Sequence sense primer sequence
cactcctggcactgcctctg

SEQ ID NO: 12 - DNA, 21 bp, Artificial Sequence
  Description of Artificial Sequence antisense primer sequence
cttgcagcccagaccatccag

SEQ ID NO: 13 - DNA, 20 bp, Artificial Sequence
  Description of Artificial Sequence sense primer sequence
ttctggtgctaatcgcagtg

SEQ ID NO: 14 - DNA, 20 bp, Artificial Sequence
  Description of Artificial Sequence antisense primer sequence
gagcagagagttgagggtgg

SEQ ID NO: 15 - DNA, 19 bp, Artificial Sequence
  Description of Artificial Sequence sense primer sequence
ggagtacctgcgcggcatg

SEQ ID NO: 16 - DNA, 21 bp, Artificial Sequence
  Description of Artificial Sequence antisense primer sequence
catggcctcggacatggacac

SEQ ID NO: 17 - DNA, 21 bp, Artificial Sequence
  Description of Artificial Sequence sense primer sequence
atgaatgagtgtcactatgac

SEQ ID NO: 18 - DNA, 21 bp, Artificial Sequence
  Description of Artificial Sequence antisense primer sequence
catacatgtagatgcgtacgt

SEQ ID NO: 19 - DNA, 21 bp, Artificial Sequence
  Description of Artificial Sequence sense primer sequence
catgcacccatgttcctgctc

SEQ ID NO: 20 - DNA, 21 bp, Artificial Sequence
  Description of Artificial Sequence antisense primer sequence
gatcggcttgcagaagcacag

(19) United States
(12) Patent Application Publication
(10) Pub. No.: US 2005/0232860 A1
(43) Pub. Date: Oct. 20, 2005

(54) Use of edg-receptor agonists for the treatment of hypertension

(76) Inventors: Gerhard J Molderings, Meckenheim (DE); Michael Bruss, Bonn (DE)

Correspondence Address: CROWELL & MORING LLP;INTELLECTUAL PROPERTY GROUP, P.O. BOX 14300, WASHINGTON, DC, 20044-4300, US

(21) Appl. No.: 10/507,583

(22) PCT Filed: Nov. 28, 2002
(86) PCT No.: PCT/EP02/13429

§ 371 Date: Sep. 13, 2004

Related U.S. Application Data
(60) Provisional application No. 60/334,106, filed on Nov. 30, 2001.

(51) Int. Cl. A61K 51/00

(52) U.S. Cl. 424/1.11

(57) ABSTRACT

Treatment and/or prophylaxis of hypertension involving administering a therapeutically active amount of an edg-receptor agonist or a pharmaceutically acceptable salt thereof. Preferably the edg-receptor agonist is a highly selective I₁-receptor agonist and essentially devoid of α₂-receptor agonist activity and has an imidazoline structure. Hence, hypertension may be treated without causing α₂-receptor induced side effects. The invention also provides appropriate screening tools for identifying compounds acting with high selectivity as I₁-receptor agonists however lacking α₂-receptor induced side effects. Furthermore, the receptor, membranes and cells comprising said receptor, and assays for screening compounds with said receptor are discussed in the context of the invention.

The present invention investigates noradrenaline release-inhibiting receptors on PC12 cells devoid of α₂- and CB₁ receptors, in particular with regard to similarities to presynaptic imidazoline and edg receptors. The invention particularly pertains to a method of treatment of hypertension, said method involving the edg-receptor. Furthermore, the receptor, membranes and cells comprising said receptor, and assays for screening compounds with said receptor are discussed in the context of the invention.

### BACKGROUND OF THE INVENTION

In addition to the “classical” presynaptic modulatory receptors on the axon terminals of postganglionic sympathetic nerves (Starke 1981), a non-I₁/non-I₂ imidazoline receptor mediating inhibition of noradrenaline release has been identified as a further presynaptic receptor in animal and human blood vessels and heart (for review, see Molderings and Göthert, 1999). This presynaptic imidazoline receptor is activated by both imidazoline and guanidine derivatives and differs pharmacologically from the presynaptic α₂-adrenoceptor. Among other criteria which differentiate between both receptors, the presynaptic imidazoline receptor is blocked with low potency by the α₂-adrenoceptor antagonist rauwolscine and with moderate potency by the CB, cannabinoid receptor antagonists SR141716A and LY320135 (Molderings and Göthert, 1998, 1999, Molderings et al., 1999).

To prove that the presynaptic imidazoline receptor is an entity independent of α₂-adrenoceptors and cannabinoid receptors, a cell line which expresses such release-inhibiting imidazoline receptors but not α₂- and CB₁ cannabinoid receptors would be helpful. The rat pheochromocytoma cell line PC12 was hypothesized to be suitable for this purpose because it possesses many properties of sympathetic neurones and adrenal chromaffin cells (for review, see Greene and Tischler, 1976). In these cells, the α₂-adrenoceptor has been reported to be absent (Schwegler and Bönisch, 1986; Duzic and Lanier, 1992); however, this is not generally accepted since other groups proposed its existence (Gatti et al., 1988; Gollasch et al., 1992; Kim et al., 1993). Therefore, the first aim of the present study was to establish the absence or presence of α₂-adrenoceptors and to examine whether PC12 cells lack CB₁ cannabinoid receptors, using radioligand binding and molecular biological techniques. The second aim was to investigate whether PC12 cells express imidazoline receptors with pharmacological properties comparable to those of the presynaptic imidazoline receptors on the sympathetic neurones of man, rat and rabbit. If so, the third aim finally was to investigate whether the release-inhibiting imidazoline receptor can be classified as a member of an already known receptor family defined by its pharmacological and/or molecular biological properties. The forth aim finally was to establish a novel method of treatment of hypertension without α₂-receptor induced side effects and to provide appropriate screening tools for identifying compounds acting with high selectivity as I₁ receptor agonists however lacking α₂-receptor induced side effects known from present antihypertensive compounds.

### SUMMARY OF THE INVENTION

According to the invention a method of treatment and/or prophylaxis of hypertension is suggested which involves the administering to a mammal a therapeutically active amount of an edg-receptor agonist or a pharmaceutically acceptable salt thereof. Preferably the invention pertains to a method of treatment and/or prophylaxis of hypertension in which the edg-receptor agonist is a highly selective I₁-receptor agonist and essentially devoid of α₂-receptor agonist activity. In particular the method of treatment and/or prophylaxis of hypertension involves an edg-receptor agonist which has an imidazoline compound structure. Hence, following the principles of the present invention shown below, hypertension may be treated and/or prophylacted by administering imidazoline compounds showing highly selective I₁ agonist activity for the edg-receptor, however without causing α₂-receptor induced side effects. These findings are supported by an experimental study. One objective of the study was to classify release-inhibiting receptors on rat pheochromocytoma PC12 cells. Veratridine-evoked [³H]noradrenaline release from PC12 cells was inhibited by μmolar concentrations of the imidazoline and guanidine derivatives cirazoline, clonidine, aganodine, 1,3-di(2-tolyl)guanidine, BDF6143 and agmatine, and of the cannabinoid receptor agonist WIN55,212-2 (R(+)-[2,3-dihydro-5-methyl-3-[(morpholinyl)methyl]pyrrolo-[1,2,3-de]-1,4-benzoxazin-yl](1-naphthalenyl)-methanone mesylate) but not by noradrenaline. The inhibitory effect of clonidine was antagonized by μmolar concentrations of rauwolscine and SR141716A (N-[piperidin-1-yl]-5-[4-chlorophenyl]-1-[2,4-dichlorophenyl]-4-methyl-1H-pyrazole-3-carboxamide). The potencies of the agonists and antagonists were compatible with an action at previously characterized presynaptic imidazoline receptors. 1-Oleoyl-lysophosphatidic acid but not sphingosine-1-phosphate, produced an inhibition of release that was antagonized by 30 μM rauwolscine, 1 μM SR141716A and 10 μM LY320135 as well as by pretreatment of the cells with 100 μM clonidine for 72 h. PCR experiments on cDNA from PC12 mRNA suggest mRNA expression of lysophospholipid receptors encoded by the genes edg2, edg3, edg5 and edg7, but not of receptors encoded by edg1, edg4, edg6 and edg8 and not of α_{2A}- and CB₁ receptors. In conclusion, PC12 cells are not endowed with α₂-adrenoceptors and CB₁ cannabinoid receptors, but with an inhibitory receptor recognizing imidazolines, guanidines and WIN55,212-2 similar to that on sympathetic nerves. The PCR results and the ability of 1-oleoyl-LPA to mimic these drugs (also with respect to their susceptibility to antagonists) suggest that the release-inhibiting receptor may be an edg-encoded lysophospholipid receptor. The invention therefore enables to a method of treatment of hypertension by involving the edg-receptor. Hence, following the principles of the present invention, hypertension may be treated and/or prophylacted by administering compounds showing highly selective I₁ agonist activity however without causing α₂-receptor induced side effects. The invention also provides appropriate screening tools for identifying compounds acting with high selectivity as I₁ receptor agonists however lacking α₂-receptor induced side effects. Furthermore, the receptor, membranes and cells comprising said receptor, and assays for screening compounds with said receptor are discussed in the context of the invention.

### BRIEF DESCRIPTION OF THE FIGURES

FIG. 1

Concentration dependence of the inhibitory effects of BDF6143 and agmatine and lack of effect of moxonidine on veratridine-induced [³H]noradrenaline release from PC12 cells. The cells were preincubated with culture medium containing 10 nM [³H]noradrenaline and, after 60-min exposure to [3H]noradrenaline-free culture medium, incubated with [³H]noradrenaline-free HEPES-buffered salt solution containing 15 mM KCl, 1 μM desipramine and 3 μM rauwolscine throughout. BDF6143, agmatine or moxonidine was present from the onset of incubation with the HEPES buffer until the end of the experiment; 5-min stimulation with 1 mM veratridine was carried out 60 min after onset of incubation with the buffer. Evoked [3H]noradrenaline release was expressed as percentage of veratridine-evoked release in control experiments carried out in parallel without the drug under study. Means±SEM of 6 experiments in each group. *P<0.05, **P<0.01 (compared with the corresponding controls).

FIG. 2

(A) Inhibitory effect of clonidine on [³H]noradrenaline release from PC12 cells and

(B) interaction with rauwolscine, SR141716A and LY320135.

The cells were preincubated with culture medium containing 10 nM [³H]noradrenaline and, after 60-min exposure to [³H]noradrenaline-free culture medium, incubated with [³H]noradrenaline-free HEPES-buffered salt solution containing 15 mM KCl and 1 μM desipramine throughout. Clonidine and the interacting drug under study (rauwolscine: 3 μM, (●); 30 μM, (▾); SR141716A: 1 μM, solid column; 10 μM, (▪); LY320135: 10 μM, dashed column) or its solvent ((∘), open column) were present from the onset of incubation with the HEPES buffer until the end of the experiment; 5-min stimulation with 1 mM veratridine was carried out 60 min after onset of incubation with the buffer. Evoked [³H]noradrenaline release was expressed as percentage of evoked release in control experiments carried out in parallel without clonidine. Means±SEM of 6 experiments in each group; *P<0.05, **P<0.01 (compared with the corresponding controls); +at least P<0.05 (compared with the effect of clonidine in the absence of interacting drugs; Dunnett's test for multiple comparisons).

FIG. 3

Effects of CB₁ receptor agonists (CP55,950, anandamide, WIN55,212-2 and WIN55,212-3) on veratridine-induced [³H]noradrenaline release from PC12 cells. The cells were preincubated with culture medium containing 10 nM [3H]noradrenaline and, after 60-min exposure to [³H]noradrenaline-free culture medium, incubated with [³H]noradrenaline-free HEPES-buffered salt solution containing 15 mM KCl, and 1 μM desipramine throughout. The agonist and antagonist under study (3 μM rauwolscine, open columns; 1 μM SR141716A+3 μM rauwolscine, solid column) were present from the onset of incubation with the HEPES buffer until the end of the experiment; 5-min stimulation with 1 mM veratridine was carried out 60 min after onset of incubation with the buffer. Evoked [³H]noradrenaline release was expressed as percentage of veratridine-evoked release in control experiments carried out in parallel without CP55,950, anandamide, WIN55,212-2 or WIN55,212-3. Means±SEM of 6 experiments in each group. * P<0.05, **P<0.01, ***P<0.001 (compared with the corresponding controls); +P<0.05 (compared with the effect of 0.3 M CP55,940 in the presence of 3 M rauwolscine).

FIG. 4

(A) Effects of 1-oleoyl-LPA and 1-palmitoyl-LPA on [³H]noradrenaline release from PC12 cells and

(B) interaction with rauwolscine, SR141716A and clonidine.

The cells were preincubated with culture medium containing 10 nM [³H]noradrenaline and, after 60-min exposure to [³H]noradrenaline-free culture medium, incubated with [³H]noradrenaline-free HEPES-buffered salt solution containing 15 mM KCl and 1 μM desipramine throughout. 1-Oleoyl-LPA and the interacting drug under study (rauwolscine: 30 μM, second solid column; SR141716A: 1 μM, third dashed column; pretreatment with 100 μM clonidine: fourth cross-hatched column) or its solvent (; first column) or 1-palmitoyl-LPA ( ) were present from the onset of incubation with the HEPES buffer until the end of the experiment; 5-min stimulation with 1 mM veratridine was carried out 60 min after onset of incubation with the buffer. Evoked [3H]noradrenaline release was expressed as percentage of evoked release in control experiments carried out in parallel without the lysophosphatidic acid derivatives. Means±SEM of 6 experiments in each group; *P>0.05, ***P<0.001 (compared with the corresponding controls); +at least P<0.05 (compared with the effect of 1-oleoyl-LPA, in the absence of interacting drugs; Dunnett's test for multiple comparisons).

FIG. 5

The results from this experiment confirming the conclusions according to the present invention are [³⁵S]GTPγS-binding to membranes from RH7777 cells stably transfected with h(edg2) receptors in response to the endogenous edg2 receptor ligand lysophospatidic acid (LPA) (FIG. 5A) and the imidazoline compounds clonidine (FIG. 5B) and moxonidine (FIG. 5C). Data are expressed as mean values+SEM of 3-7 experiments in each series. Maximum stimulation of [³⁵S]GTPγS amounted to 500-1500 dpm on average.

### DETAILED DESCRIPTION OF THE INVENTION

According to the invention a method of treatment and/or prophylaxis of hypertension is suggested which involves the administering to a mammal a therapeutically active amount of an edg-receptor agonist or a pharmaceutically acceptable salt thereof. Preferably the invention pertains to a method of treatment and/or prophylaxis of hypertension in which the edg-receptor agonist is a highly selective I₁-receptor agonist and essentially devoid of α₂-receptor agonist activity. In particular the method of treatment and/or prophylaxis of hypertension involves an edg-receptor agonist which has an imidazoline compound structure. Hence, following the principles of the present invention shown below, hypertension may be treated and/or prophylacted by administering imidazoline compounds showing highly selective I₁ agonist activity for the edg-receptor, however without causing α₂-receptor induced side effects.

For the treatment and/or prophylaxis of hypertension in accordance with the invention, the edg-receptor agonist or its physiologically compatible salt, e.g. acid-addition salts, any suitable route of administration may be employed, i.e. the compound can be administered orally, intravenously or transdermally in conventional pharmaceutical preparations.

For example, according to the invention the therapeutically active quantities of the compounds that are used for the treatment and/or prophylaxis of hypertension can be contained together with customary pharmaceutical excipients and/or additives in solid or liquid pharmaceutical formulations. Examples of solid dosage forms are tablets, coated tablets, capsules, powders, granules or suppositories. These solid dosage forms can contain standard pharmaceutical inorganic and/or organic excipients such as lactose, talc or starch in addition to customary pharmaceutical additives such as lubricants or tablet disintegrants. Liquid preparations such as solutions (i.e. solutions to be administered iv) suspensions or emulsions of the active ingredients can contain the usual diluents such as water, oil and/or suspending aids such as polyethylene glycols and the like. Further additives such as preservatives and the like may also be added.

The active ingredients can be mixed and formulated with the pharmaceutical excipients and/or additives in a known manner. For the manufacture of solid dosage forms, for example, the active ingredients may be mixed with the excipients and/or additives in the usual manner and granulated in a wet or dry process. Granules or powder can be filled directly into capsules or compressed into tablet cores. If desired, these can be coated in a known manner. For the manufacture of liquid dosage forms the active compounds are dissolved in a suitable liquid carrier and optionally suitable adjuvants may be added.

Pharmaceutical compositions suitable for injection (i.e. for iv administration) may be sterilised solutions containing a therapeutically active amount of an edg-receptor agonist or its physiologically compatible salt dissolved in a physiologically acceptable isotonic saline solution (i.e., containing 0.9% by wt. sodium chloride). Usually these solutions are adopted in a known manner to the physiological characteristics of the site of administration.

Furthermore, the edg-receptor, membranes and cells comprising said receptor, and assays for screening compounds with said receptor, which are suitable to treat hypertension with substantially reduced side effects, are described in the context of the invention.

The findings of the present invention are based on the following tests and study results.

Test and Results

In the present study we investigated whether PC12 cells, which represent a neuronal-like rat pheochromocytoma cell line, are endowed with release-inhibiting receptors that resemble previously characterized presynaptic imidazoline receptors on sympathetic nerves (Molderings and Göthert 1999), but lack α₂-adrenoceptors and cannabinoid CB, receptors. The ultimate aim was to investigate whether the release-inhibiting receptors can be classified as belonging to a receptor family which has already been defined by its pharmacological and molecular properties.

Our PCR experiments with primer pairs flanking the coding region of the rα_{2A}-adrenoceptor gene on cDNA prepared from undifferentiated PC12 cells revealed that PC12 cells do not express α_{2A}-adrenoceptors which have been proved to act as presynaptic autoreceptors (Trendelenburg et al., 1997). This finding is in accordance with the lack of specific high affinity binding of [3H]rauwolscine (Duzic and Lanier, 1992; present study), but appears to be in contrast to the results of functional studies on undifferentiated PC12 cells (Gatti et al., 1988; Gollasch et al., 1992; Kim et al., 1993), in which clonidine was used as a purported selective α₂-adrenoceptor agonist (Gatti et al., 1988) or yohimbine acted as an antagonist at the very high concentration of 100 μM (Gollasch et al., 1992; Kim et al., 1993). However, these drugs (in particular at the concentrations used) are by no means selective α₂-adrenoceptors ligands; thus, the results described by those authors are in line with the data of the present study and are compatible with our final conclusions (see below). The failure to detect specific high affinity [³H]rauwolscine binding which labels α_{2A}-, α_{2B}- and α_{2C}-adrenoceptors, and mRNA for α_{2A}-adrenoceptors in undifferentiated PC12 cells provides clear evidence that these cells are not endowed with any subtype of α₂-adrenoceptors.

In view (1) of the ability of PC12 cells to produce the endogenous cannabinoid receptor agonist anandamide (Bisogno et al., 1998) and to exhibit responses to cannabinoid receptor ligands (Tahir et al., 1992; Kiss, 1999) and (2) of the putative relationship of the presynaptic imidazoline receptor to (but not identity with) the CB₁ cannabinoid receptor (Molderings et al., 1999), it was of importance to examine whether PC12 cells express CB₁ receptors. Our results from the PCR experiments with primers for the rat CB₁ receptor gene suggest that undifferentiated PC12 cells do not express detectable amounts of CB₁ receptors. This conclusion is supported by the failure to demonstrate specific high affinity binding of [³H]SR141716A, a highly selective CB, receptor radioligand to membranes from undifferentiated PC12 cells.

In order to identify receptors on PC12 cells with the characteristic features of presynaptic imidazoline receptors, veratridine-evoked tritium overflow from PC12 cells preincubated with [³H]noradrenaline was determined, which under the present conditions (blockade of the neuronal noradrenaline transporter) reflects release of tritiated and endogenous noradrenaline from the PC12 cells (for details, see Bönisch et al. 1990). Veratridine stimulates Na⁺ influx mainly through voltage-dependent Na⁺ channels (Ulbricht, 1998). Since [³H]noradrenaline release was completely suppressed by omission of Ca²⁺ ions and markedly reduced by colchicine (which impairs the microtubular system involved in exocytosis), veratridine-induced [³H]noradrenaline release from PC12 cells resembles action-potential-induced exocytotic [³H]noradrenaline release from sympathetic neurones. Undifferentiated PC12 cells, i.e. cells not treated with nerve growth factor, express only a low number of veratridine-sensitive Na⁺ channels (Reed and England 1986; Rudy et al. 1987). To elicit a detectable and reproducible noradrenaline release by veratridine, the signal had to be increased by partial depolarization with elevated K⁺ (15 mM).

The present results prove that PC12 cells express receptors with the characteristic features of presynaptic imidazoline receptors mediating inhibition of noradrenaline release in cardiovascular tissue (Molderings and Göthert, 1999; Molderings et al. 1999): (1) Moxonidine and the catecholamine noradrenaline which stimulate presynaptic α₂-adrenoceptors but not presynaptic imidazoline receptors, were inactive in the present experiments on PC12 cells; in contrast, aganodine and clonidine which are preferential agonists at presynaptic imidazoline receptors inhibited [³H]noradrenaline release in the present study. (2) Further imidazoline and guanidine derivatives such as cirazoline, BDF6143 and DTG also inhibited release irrespective of whether or not they possess affinity as agonists or antagonists for α₂-adrenoceptors. Their rank order of potency in inhibiting [³H]noradrenaline release from PC12 cells was similar to that in cardiovascular tissue. (3) Rauwolscine acted as an antagonist versus clonidine at the imidazoline recognizing receptors but with markedly lower potency than at α₂-adrenoceptors. (4) The highly selective CB₁ cannabinoid receptor antagonists SR141716A (Rinaldi-Carmona et al., 1995) and LY320135 (Felder et al., 1998) counteracted the inhibitory effect of clonidine but with potency lower than at CB₁ receptors.

The cannabinoid receptor agonist WIN55,212-2 also produced a low potency inhibition of [³H]noradrenaline release, whereas WIN55,212-3, an enantiomer which is inactive at CB₁ cannabinoid receptors but shares the lipophilicity of WIN55,212-2, did not. The cannabinoid receptor agonist CP55,940 at a concentration of 0.3 μM also inhibited [³H]noradrenaline release, an effect which was antagonized by 1 μM SR141716A. The more the concentration of CP55,940 was increased above 0.3 μM, the more the inhibitory effect disappeared and was finally reversed to a facilitation. Anandamide, an endogenous agonist at cannabinoid receptors, also facilitated [³H]noradrenaline release from PC12 cells. Similarly, a facilitation of [³H]noradrenaline release has previously been observed in the human heart with CP55,940 and anandamide (Molderings et al., 1999), when LY320135 was present in the superfusion fluid. In conclusion, the results with the cannabinoid receptor agonists suggest that these drugs may activate not only a release-inhibiting but also a release-facilitating receptor. The latter is unmasked on PC12 cells and sympathetic nerves by increasing the agonist concentration and addition of a CB₁ cannabinoid receptor antagonist, respectively; no attempt has been made in this study to classify this receptor.

Taken together, the receptors mediating inhibition of [³H]noradrenaline release in cardiovascular tissue and PC12 cells resemble each other with respect to their pharmacological properties and share properties with the α₂-adrenoceptor and the CB₁ cannabinoid receptor; however, they are not identical with those receptors because the expression of α₂- and CB₁ cannabinoid receptors on PC12 cells was ruled out by our radioligand binding and PCR experiments. A comparison of the nucleotide sequences of the α₂-adrenoceptors and the CB₁ cannabinoid receptor with those of G-protein-coupled receptors listed in the gene databases revealed that only lysophospholipid receptors possess a significant homology with both receptor classes (about 40%). Accordingly, 1-oleoyl-LPA (but not S1P) inhibited evoked [³H]noradrenaline release and this effect was antagonized at low potency by rauwolscine, SR141716A and LY320135. Moreover, pretreatment of the cells for 72 hours with 100 μM clonidine which has been assumed to desensitize the presynaptic imidazoline receptors (Göthert and Molderings 1991; Fuder and Schwarz 1993), abolished the inhibitory effect of 1-oleoyl-LPA. This finding indicates that 1-oleoyl-LPA and at least clonidine may exert their inhibitory effect via the same receptor. On the other hand, similar to the cannabinoids CP55,940 and anandamide (see above) 1-palmitoyl-LPA which has been shown to be able to discriminate between the LPA-sensitive lysophospolipid receptors (Bandoh et al. 1999; Im et al. 2000), up to 1 μM increased evoked [³H]noradrenaline release; at 10 μM the facilitation appeared to be counterbalanced by a simultaneously elicited inhibitory effect. Higher concentrations to study the latter putative inhibitory effect could not be investigated because vehicle-induced effects made an exact evaluation impossible.

The ability of 1-oleoyl-LPA to mimic the pharmacological properties of the imidazoline and guanidine derivatives at the release-inhibiting receptor points to the possibility that this receptor may represent a lysophospholipid receptor. At present eight lysophospholipid receptor types have been identified and cloned. They are provisionally termed edg1-8 according to the endothelial differentiation genes 1-8 which code for these receptors. 1-Oleoyl-LPA is an agonist at edg2, edg4 and edg7, whereas it is almost not active at edg1, edg3, edg5, edg6 and edg8. In contrast, S1P is an agonist at the latter receptors, whereas it is inactive at the former ones. Using the PCR technique, no cDNA coding for edg1, edg4, edg6 and edg8 receptors was detectable in PC12 cells. However, we demonstrated the presence of cDNAs encoding edg2, edg3, edg5 and edg7 receptors in PC12 cells. Hence, these results of the PCR experiments together with those of our release experiments and with the pattern of edg receptors that are activated by 1-oleoyl-LPA (see above) are at least compatible with the possibility that the release-inhibiting receptor may be identical with the edg2 or the edg7 receptor or a so far unknown lysophospholipid receptor at which 1-oleoyl-LPA acts as an agonist.

In conclusion, the three questions addressed in the Introduction can be answered as follows: (1) In contrast to the argumentation by Gatti et al. (1988), Gollasch et al. (1992) and Kim et al. (1993), our data from [3H]rauwolscine binding and PCR experiments unequivocally provide evidence that PC12 cells are not endowed with α₂-adrenoceptors. Also, as proved by bindung and PCR techniques, these cells are devoid of CB₁ receptors. (2) The present [³H]noradrenaline release experiments revealed that the release-inhibiting receptor on undifferentiated PC12 cells exhibits the same pharmacological properties (such as low potency activation by imidazolines, guanidines and CB, receptor agonists and low potency blockade by rauwolscine and CB₁ receptor antagonists) as the release-inhibiting receptor on sympathetic nerve terminals previously denoted as presynaptic non-I₁/non-I₂-imidazoline receptor (Molderings and Göthert, 1999). In view of the absence not only of α₂-adrenoceptors but also of CB₁ receptors in PC12 cells, the identity of the pharmacological properties suggests that the release-inhibiting receptor may be identical to the cardiovascular presynaptic imidazoline receptor. (3) Since 1-oleoyl-LPA also inhibits noradrenaline release from PC12 cells in a manner sensitive to receptor desensitization by clonidine and to low potency antagonism by CB₁ receptor antagonists and rauwolscine, 1-oleoyl-LPA may also act via such a so-called imidazoline receptor. Furthermore, PC12 cells were found to express mRNA for 1-oleoyl-LPA-activated edg2 and edg7 receptors. Taken together, these results are compatible with the possibility that the release-inhibiting receptors so far known as presynaptic non-I₁/non-I₂-imidazoline receptors are edg-encoded lysophospholipid receptors. More direct support for this conclusion can be given by experiments on cells transfected with cDNAs coding for edg receptors.

Screening of Compounds

The invention may also be used to screen for compounds for the treatment of hypertension.

Accordingly the invention also pertains to a method for determining or identifying whether a substance, preferably a candidate compound, is a potential ligand of an edg-encoded lysohospholipid receptor, preferably of a mammalian edg-encoded lysohospholipid receptor, wherein said method comprises:
a. contacting cells expressing the edg-encoded lysohospholipid receptor polypeptide, or contacting a receptor membrane preparation comprising said edg-encoded lysohospholipid receptor polypeptide, with a labeled ligand, such as described in the specification, in the presence and in the absence of the substance; and
b. measuring the binding of the labeled ligand to the edg-encoded lysohospholipid receptor.

Alternatively the invention also pertains to a method for determining or identifying whether a substance, preferably a candidate compound, modulates the interaction of ligand, such as described in the specification, with edg-encoded lysohospholipid receptor polypeptide, preferably with a mammalian edg-encoded lysohospholipid receptor polypeptide, wherein said method comprises:
a. contacting cells expressing on the surface thereof an edg-encoded lysohospholipid receptor polypeptide, said receptor being associated with a second component capable of providing a detectable signal in response to the binding of a compound to said receptor, with a substance, preferably with a candidate compound, to be screened under conditions to permit binding to the receptor; and
b. determining whether the substance, preferably the candidate compound, modulates the interaction a ligand, such as described in the specification, and the edg-encoded lysohospholipid receptor polypeptide by detecting an increase or decrease in the signal normally generated by the interaction of the ligand, such as described in the specification, with the receptor after interaction of the ligand with the receptor.

The invention also pertains to a method for determining or identifying whether a substance, preferably a candidate compound, inhibits or antagonizes the interaction of a ligand, such as described in the specification, with an edg-encoded lysohospholipid receptor polypeptide, preferably with a mammalian edg-encoded lysohospholipid receptor polypeptide, wherein said method comprises:
a. contacting cells expressing on the surface thereof an edg-encoded lysohospholipid receptor polypeptide, said receptor being associated with a second component capable of providing a detectable signal in response to the binding of a compound to said receptor, with a substance, preferably with a candidate compound, to be screened under conditions to permit binding to the receptor; and
b. determining whether the substance, preferably the candidate compound, inhibits or antagonizes the interaction of the ligand, such as described in the specification, and the edg-encoded lysohospholipid receptor polypeptide by detecting an increase or decrease in the signal normally generated by the interaction of the ligand, such as described in the specification, with the receptor after interaction of the ligand with the receptor.

Furthermore, the invention pertains to a method for determining or identifying whether a substance, preferably a candidate compound, is an agonist to an edg-encoded lysohospholipid receptor polypeptide, preferably to a mammalian edg-encoded lysohospholipid receptor polypeptide, wherein said method comprises:
a. contacting cells expressing on the surface thereof an edg-encoded lysohospholipid receptor polypeptide with a substance, preferably a candidate compound; and
b. determining whether the substance, preferably the candidate compound, effects a signal generated by activation of the edg-encoded lysohospholipid receptor polypeptide, using a ligand, such as described in the specification, as a positive control for the generation of a signal.

In addition the invention also pertains to a method for determining or identifying whether a substance, preferably a candidate compound, is a modulator, an agonist or antagonist to a noradrenaline release-inhibiting edg-encoded lysohospholipid receptor polypeptide, preferably to a mammalian edg-encoded lysohospholipid receptor polypeptide, wherein said method comprises:
a. loading cells expressing on the surface thereof an edg-encoded lysohospholipid receptor polypeptide with labelled noradrenaline, then
b. contacting the labelled noradrenaline loaded cells expressing on the surface thereof an edg-encoded lysohospholipid receptor polypeptide with a substance, preferably a candidate compound,
c. adding veratridine to the labelled noradrenaline loaded cells expressing on the surface thereof an edg-encoded lysohospholipid receptor polypeptide cells for evoking noradrenaline-release in the presence of the substance, preferably in the presence of the candidate compound, and
d. determining whether the interaction of the substance, preferably the candidate compound, with the edg-encoded lysohospholipid receptor polypeptide modulates, agonizes or antagonizes the release of noradrenaline.

In particular the invention pertains to a screening method as described before, wherein the substance, preferably the candidate compound, is effective with regard to dysfunctions, disorders or diseases of the cardiovascular system, including the heart, of the nervous system, including the central nervous system, and also of glucose and insulin metabolism or with regard to dysfunctions, disorders or diseases related to increased sympathetic tonicity. Preferably the substance, more preferably the candidate compound, is effective with regard to dysfunctions, disorders or diseases associated with the cardiovascular system, preferably including blood pressure control, e.g. hypertension or vasodilatation, myocardial ischaemia, ischaemic preconditioning, cardioprotective activity, or other heart related diseases.

In the screening methods according to the invention isolated rat pheochromocytoma PC12 cells comprising an edg-encoded lysohospholipid receptor may be used. The isolated rat pheochromocytoma PC12 cells may be used comprising an edg-encoded receptor which is a nordrenaline release-inhibiting receptor. Preferably, in the screening methods according to the invention isolated rat pheochromocytoma PC12 cells are used comprising a receptor which is encoded by the genes edg2, edg3, edg5 and/or edg7. Preferably in the screening methods of the invention the isolated rat pheochromocytoma PC12 cells preferably are devoid of α₂-receptors and/or CB₁-receptors.

The experimental data and results of the foregoing described study suggest that the edg receptor is not only binding imidazoline compounds but that after binding the edg receptor responds with activation. Hence the edg receptor may be used for activation by imidazoline compounds e.g. for the treatment and/or prophylaxis of hypertension or optionally also for the treatment and/or prophylaxis of other dysfunctions, disorders or diseases of the cardiovascular system, e.g. as those stated supra. It is a particular advantage of the invention that by using the edg receptor in screening methods imidazoline compounds may be provided that are highly I₁ selective with essentially reduced or even no α₂-indiuced side effects. This provides a substantial improvement over the state of the art, e.g. it is possible to avoid α₂-induced side effects like sedation as known from the α₂-/I₁-compound clonidine.

### EXAMPLES

The following examples are merely intended to illustrate the invention, but without limiting the invention.

1. Experimental Procedures

Cell Culture

PC12 cells were cultured in suspension culture as described by Harder and Bönisch (1984). The culture medium was composed of 85% RPMI 1640 medium (Gibco, Karlsruhe, FRG), 10% heat-inactivated horse serum (Gibco), 5% fetal calf serum (Gibco) and was buffered with 24 mM NaHCO₃. For the experiments on intact cells, PC12 cells were cultured in a humidified CO₂-incubator (at 37° C. and in the presence of 9% CO₂) on dishes (60 mm, Nunc) coated with polyornithine 0.1 g/l (in 0.15 M boric acid, and 67 mM NaOH, adjusted with HCl to pH 8.4). Each culture dish contained about 10⁷ cells corresponding to about 1 mg cell protein.

Membrane Preparation

Undifferentiated PC12 cells were harvested and homogenized in HEPES buffer (HEPES-Na⁺ 5 mM, EGTA 0.5 mM, MgCl₂ 0.5 mM, ascorbic acid 0.1 mM, pH 7.4, 4° C.) with a polytron homogenizer for 1 min and then centrifuged for 10 min (1000×g; 4° C.) to pellet cellular debris. The supernatant was centrifuged for 30 min (40000×g; 4° C.) and the pellet resuspendend in fresh HEPES buffer and washed twice. Finally, the washed pellet was stored at −80° C. until use. Before the membranes were added to the incubation assay, they were centrifuged (20 min, 40000 g, 4° C.), resuspended in the buffer, homogenized by ultrasonication and diluted to a final protein concentration of about 0.6 mg/ml.

Radioligand Binding

A 400-μl aliquot of the membranes was incubated for 30 min with 16.8 nM [³H]rauwolscine or for 60 min with 10 nM [³H]SR141716A at room temperature in a final volume of 0.5 ml. The reaction was stopped by rapid vacuum filtration with a Brandel cell harvester through Whatman GF/C glass-fiber filters presoaked with polyethylenimine 0.5 M followed by rapid washing of the incubation tubes and filters with 10 ml ice-cold buffer. Filters were placed in 6 ml of scintillation fluid and shaken overnight, and the radioactivity was determined by liquid scintillation counting at 44% efficiency. Non-specific binding was defined as [³H]rauwolscine binding in the presence of 10 μM rauwolscine and as [³H]SR141716A binding In the presence of 3 μM CP55,940. Results are expressed as mean values±SEM. All experiments were carried out in triplicate. Data were analyzed using the least-squares fitting program PRISM (GraphPad Software Inc., San Diego, USA).

PCR Amplification of α_{2A}-, CB₁ and Lysophospholipid Receptor DNA

Template DNAs were first strand cDNA from undifferentiated PC12 cells and genomic DNA prepared from rat whole blood. To prepare cDNA, cells were chemically dissected; total RNA from PC12 cells was isolated with the Quiagen RNeasy kit according to the manufacturers protocol. Then oligo dT primed first strand cDNA synthesis was performed with AMV Reverse Transcriptase (Promega), followed by RNase digestion, phenol/chloroform extraction and cDNA purification with Chroma Spin 30 columns (Clontech). Genomic DNA was purified from rat whole blood with the Quiagen QIAamp Blood Midi kit according to the manufactorer's protocol. The purified DNA was used as template for subsequent PCR amplification of the receptor DNA under the following conditions: primer sequences for the rat α_{2A}-adrenoceptor [sense primer: 5′-catctccttcccgccactcatc-3′; antisense primer: 5′-atacgcacgtagaccaggatc-3′] and for the rat CB₁ cannabinoid receptor [sense primer: 5′-ctggc(ac)(gt)(ag)gc(agct)gac(agct)tcctg-3′; antisense primer: 5-a(gt)(ag)g(ct)(ag)tagat(agct)a(agct) (agct)gggttc-31 were chosen according to the sequences of Gene Bank acc. no. M62372 and acc. No. X81120, respectively. Primer sequences for the lysophospholipid receptors were chosen according to the following sequences: edg1: acc. no. U10303 (sense primer: 5′-cttcagcctccttgctatcg-3′; antisense primer: 5′-gcaggcaatgaagacactca-3′); edg2: acc. no. AF014418 (sense primer: 5′-ccaaacta cagcactctcatg-3′; antisense primer: 5′-gcttccttctaaaccacagag-3′); edg3: acc. no. NM005226 (sense primer: 5′-tcagggagggcagtatgttc-3′; antisense primer: 5′-ctgactttcgaagaggatgg-3′); edg4: acc. no. AW477544 (sense primer: 5′-cactcctggcactgcctctg-3′; antisense primer: 5′-cttgcagccc agaccatccag-3′); edg5: acc. no. U10699 (sense primer: 5′-ttctggtgctaatcgcagtg-3′; antisense primer: 5′-gagcagagagttgagggtgg-3′); edg6: acc. no. AW141943 (sense primer: 5′-ggagtacctgcgcggcatg-3′; antisense primer: 5′-catggcctcggacatggacac-3′); edg7: acc. no. AW107032 (sense primer: 5′-atgaatgagtgtcactatgac-3′; antisense primer: 5′-catacatgtagatgcgtacgt-3); edg8: acc. no. AF233649 (sense primer: 5′-catgcacccatgttcctgctc-3′; antisense primer: 5′-gatcggcttgcagaagcacag-3′). PCR was performed in a total volume of 100 μl containing 15 nM primer (each), 5 U Taq DNA Polymerase (Gibco), 2 mM MgCl₂, 200 μM dNTPs (each), 10 μl 10×Taq-Buffer (Gibco) and 3-5 μl template DNA. PCR was performed for 37-40 cycles. PCR products were separated by agarose gel electrophoresis, and the band of interest was cut off the gel, purified with GeneElute” columns (Supelco), ligated into the TA-cloning” vector pCR2.1 (Invitrogen) and transformed into E. coli InV (Invitrogen). The subcloned DNA fragments were sequenced with an automated sequencer (Li-COR 4200, MWG-Biotech, Ebersberg, Germany) and the Thermo Sequenase fluorescent labelled primer cycle sequencing kit with 7-deaza-dGTP (Amersham, Freiburg, Germany).

Summary Table of Primer Sequences

| | |
|---|---|
| Sequence | Description |

| | | |
|---|---|---|
| SEQ ID NO: 1 | 5′-catctccttcccgccactcatc-3′ | rat α_{2A}-adrenoceptor sense primer |
| SEQ ID NO: 2 | 5′-atacgcacgtagaccaggatc-3′ | rat α_{2A}-adrenoceptor antisense |
| | | primer |
| SEQ ID NO: 3 | 5′-ctggc(ac)(gt)gc(agct)gac(agct)tcctg-3′ | rat CB₁ cannabinoid receptor sense |
| | | primer: |
| SEQ ID NO: 4 | 5-a(gt)(ag)g(ct)(ag)tagat(agct)a(agct)(agct)gggttc-3′ | rat CB₁ cannabinoid receptor |
| | | antisense primer |
| SEQ ID NO: 5 | 5′-cttcagcctccttgctatcg-3′ | edg1 lysophospholipid sense primer |
| SEQ ID NO: 6 | 5′-gcaggcaatgaagacactca-3′ | edg1 lysophospholipid antisense |
| | | primer |
| SEQ ID NO: 7 | 5′-ccaaacta cagcactctcatg-3′ | edg2 lysophospholipid sense primer |
| SEQ ID NO: 8 | 5′-gcttccttctaaaccacagag-3′ | edg2 lysophospholipid antisense |
| | | primer |
| SEQ ID NO: 9 | 5′-tcagggagggcagtatgttc-3′ | edg3 lysophospholipid sense |
| | | primer |
| SEQ ID NO: 10 | 5′-ctgactttcgaagaggatgg-3′ | edg3 lysophospholipid antisense |
| | | primer |
| SEQ ID NO: 11 | 5′-cactcctggcactgcctctg-3′ | edg4 lysophospholipid sense primer |
| SEQ ID NO: 12 | 5′-cttgcagccc agaccatccag-3′ | edg4 lysophospholipid antisense |
| | | primer |
| SEQ ID NO: 13 | 5′-ttctggtgctaatcgcagtg-3′; | edg5 lysophospholipid sense primer |
| SEQ ID NO: 14 | 5′-gagcagagagttgagggtgg-3′ | edg5 lysophospholipid antisense |
| | | primer |
| SEQ ID NO: 15 | 5′-ggagtacctgcgcggcatg-3′ | edg6 lysophospholipid sense primer |
| SEQ ID NO: 16 | 5′-catggcctcggacatggacac-3′ | edg6 lysophospholipid antisense |
| | | primer |
| SEQ ID NO: 17 | 5′-atgaatgagtgtcactatgac-3′ | edg7 lysophospholipid sense primer |
| SEQ ID NO: 18 | 5′-catacatgtagatgcgtacgt-3′ | edg7 lysophospholipid antisense |
| | | primer |
| SEQ ID NO: 19 | 5′-catgcacccatgttcctgctc-3′ | edg8 lysophospholipid sense primer |
| SEQ ID NO: 20 | 5′-gatcggcttgcagaagcacag-3′ | edg8 lysophospholipid antisense |
| | | primer |

Release Experiments

To load the cells with labelled noradrenaline, 10 nM [³H]noradrenaline was added to the culture medium (3 ml/dish) and the cells were incubated for 3 h in a CO₂-incubator (37° C.). To minimize oxidation of the catecholamine, L(+)-ascorbic acid (1 mM) was present in all solutions. The cells were then washed twice with culture medium and incubated for 60 min with [³H]noradrenaline-free culture medium (with a change of the medium after 30 min). Subsequently the cell dishes were transferred to a water bath of 37° C. (onset of the experiment) and the cells were then incubated until the end of the experiment with prewarmed (37° C.) [³H]noradrenaline-free HEPES-buffered salt solution (composition in mM unless stated otherwise: NaCl 125, KCl 15, KH₂PO₄ 1.2, CaCl₂ 2.6, MgSO₄ 1.2, HEPES-NaOH (pH 7.4) 25, D(+)-glucose 5.6, L(+)-ascorbic acid 1.0) with a change of the medium every 5 min (total incubation time in the HEPES buffer: 85 min). To minimize loss of cells during washing und to inhibit the neuronal noradrenaline transport system the solution contained 1 g/l bovine serum albumin and 1 μM desipramine, respectively (Friedrich and Bönisch, 1986). The agonist and/or antagonist under study was added to the HEPES buffer at the onset of the exposure to this buffer. Sixty min after onset of incubation with the buffer 1 mM veratridine was added for 5 min. In a few experiments, the HEPES-buffered salt solution contained 4.8 instead of 15 mM KCl and stimulation was carried out either by 1 mM veratridine or by increasing the KCl concentration to 15 mM (time schedule identical to the standard procedure). In one series of experiments, culture medium was changed daily (instead of changing it every 3 days as standard condition) and 100 μM clonidine was added to the medium for 72 h. The cells were then washed twice with clonidine-free culture medium (with a change of the medium after 30 min). Subsequently the cell dishes were transferred to a water bath of 37° C. (onset of the experiment) and the cells were then incubated until the end of the experiment with prewarmed (37° C.) [³H]noradrenaline-free HEPES-buffered salt solution and veratridine was administered 2 h after the end of exposure to clonidine. Basal efflux of tritium was determined in the 5-min period before and in that 20 min after onset of stimulation. Veratridine-evoked tritium overflow was determined in the four 5-min periods after the onset of veratridine application. At the end of the experiment, the cells were solubilized by 0.1% v/v TritonX-100 (in 5 mM Tris-HCl, pH 7.4). The radioactivity of the solubilized cells und of the wash-out samples was determined by liquid scintillation counting.

Materials

Rauwolscine hydrochloride, agmatine sulfate, noradrenaline base, 1-oleoyl-lysophosphatidic acid, sphingosine-1-phosphate (Sigma, Munich, FRG); cirazoline hydrochloride (Synthélabo, Paris, France); aganodine, moxonidine, 4-chloro-2-(2-imidazolin-2-ylamino)-isoindoline hydrochloride (BDF 6143; Beiersdorf, Hamburg, FRG); desipramine hydrochloride (Ciba-Geigy, Wehr, Germany); clonidine hydrochloride (Boehringer, Ingelheim, FRG); N-[piperidin-1-yl]-5-[4-chlorophenyl]-1-[2,4-dichlorophenyl]-4-methyl-1H-pyrazole-3-carboxamide (SR141716A; Sanofi, Montpellier, France); [(−)-cis-3-[2-hydroxy-4-(1,1-dimethylheptyl)phenyl]-trans-4-(3-hydroxypropyl)-cyclohexane (CP55,940; Tocris-Cookson-Biotrend, Cologne, FRG); [6-methoxy-2-(4-methoxyphenyl)benzo[b]thien-3-yl][4-cyanophenyl]methanone (LY320135; Lilly, Indianapolis, USA); 1,3-di(2-tolyl)guanidine (DTG), anandamide (RBI, Natick, USA); R(+)-[2,3-dihydro-5-methyl-3-[(morpholinyl)methyl]pyrrolo[1,2,3-de]-1,4-benzoxazin-yl)(1-naphthalenyl)methanone mesylate (WIN55,212-2), WIN55,212-3 (S(−)enantiomer of WIN55,212-2, (RBI-Biotrend, Cologne, FRG); 1-palmitoyl-LPA (Avanti Polaris Lipids, Alabaster, USA); (−)-[2,5,6-³H]noradrenaline (specific activity 46 Ci/mmol, NEN, Dreieich, FRG); [O-methyl-3H]rauwolscine (specific activity 76 Ci/mmol), [³H]SR141716A (specific activity 44.0 Ci/mmol, Amersham, Braunschweig, FRG). Drugs were dissolved in saline with the following exceptions: DTG was dissolved in methanol; SR141716A, LY320135, anandamide, WIN55,212-2, WIN55,212-3, CP55,940, 1-oleoyl-LPA, 1-palmitoyl-LPA and S1P were dissolved in dimethylsulfoxide (final maximum concentration in the buffer solution 1%). The stock solutions were further diluted in the buffer. Corresponding control experiments were run with the solvent only.

2. Results

[³H]Noradrenaline Release Experiments

At a KCl concentration of 4.8 mM in the incubation buffer (i.e. the KCl concentration in most classical physiological salt solutions), 1 mM veratridine induced only an inconsistant and small tritium overflow above basal outflow from PC12 cells preincubated with [³H]noradrenaline (n=18; not shown). When the KCl concentration was increased to 15 mM for 5 min after 60 min exposure to 4.8 mM KCl in the incubation buffer also no consistent tritium overflow occurred (n=5; not shown). However, in the presence of 15 mM KCl in the incubation buffer, 1 mM veratridine induced a tritium overflow above basal efflux which in a representative series of control experiments amounted to 1.39±0.29% of the tritium present in the cells before the respective collection period (n=24; corresponding to 1760±285 dpm/well). This veratridine (1 mM)-evoked tritium overflow was abolished in the absence of Ca²⁺ ions in the incubation buffer (n=6); it was inhibited by 56% by 50 μM colchicine (n=6). In all further experiments, the veratridine stimulus in the presence of 15 mM KCl was applied as the standard stimulation procedure with this depolarizing alkaloid. The veratridine-evoked tritium overflow reflects exocytotic release of tritiated and unlabelled noradrenaline (for details, see Discussion) and is denoted as [³H]noradrenaline release in the following text, table and figures.

The basal tritium efflux from the cells incubated with the buffer containing 15 mM KCl during the 5-min period before stimulation with veratridine amounted to 1.31±0.10% of the tritium present in the cells before this collection period corresponding to 593±71 dpm/well (n=24 in a representative series of control experiments). It was not altered by the antagonists or agonists investigated in this study.

Effect of Imidazoline and Guanidine Derivatives and Interaction with Antagonists

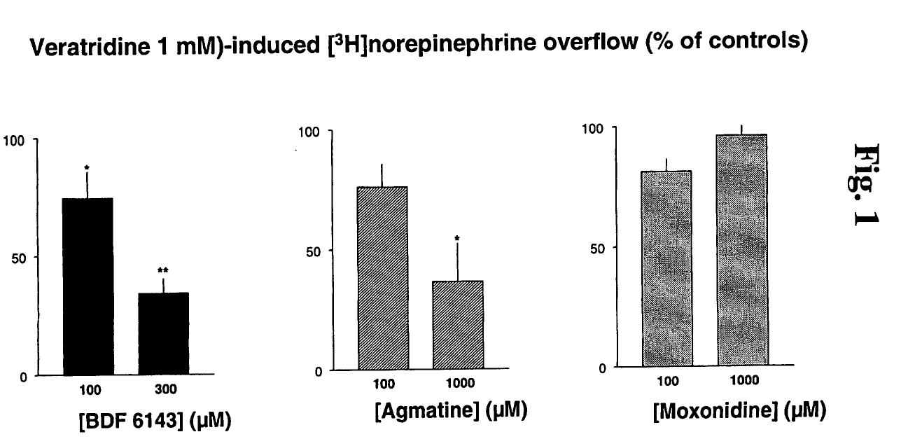

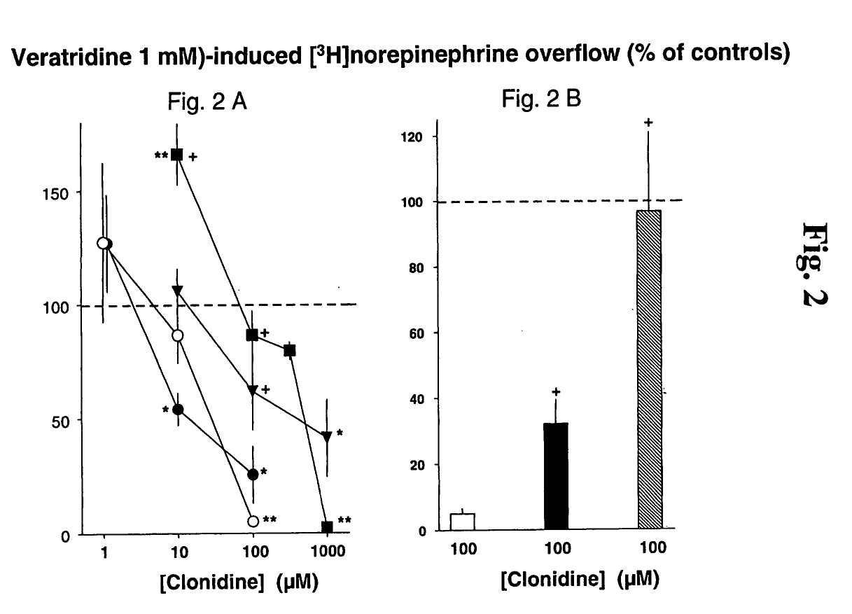

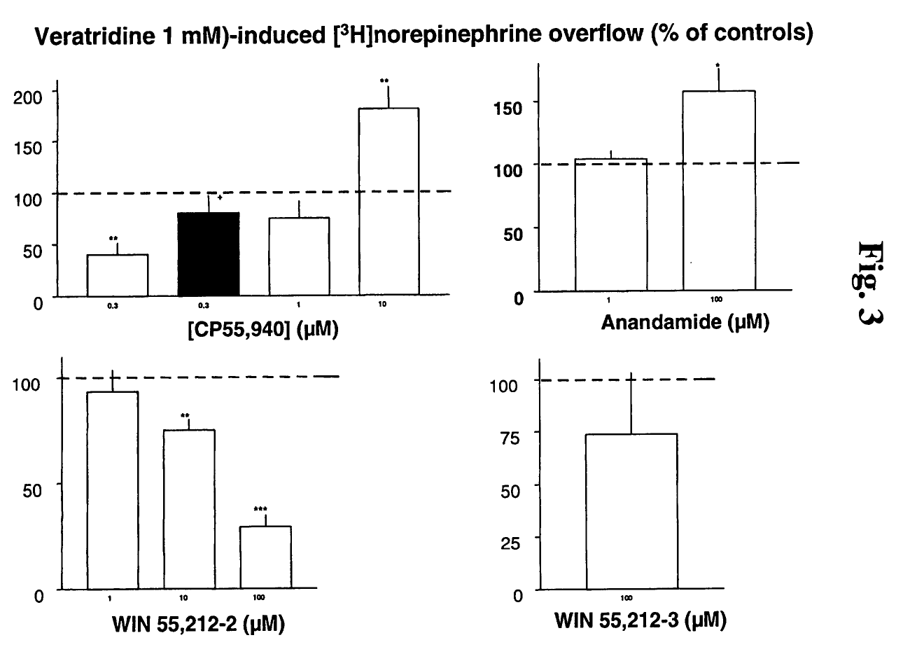

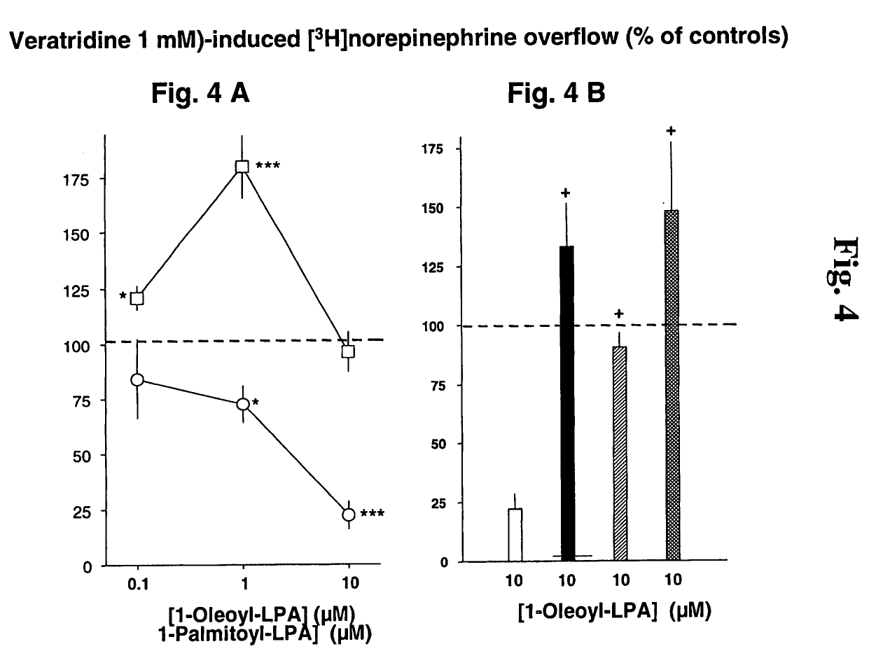

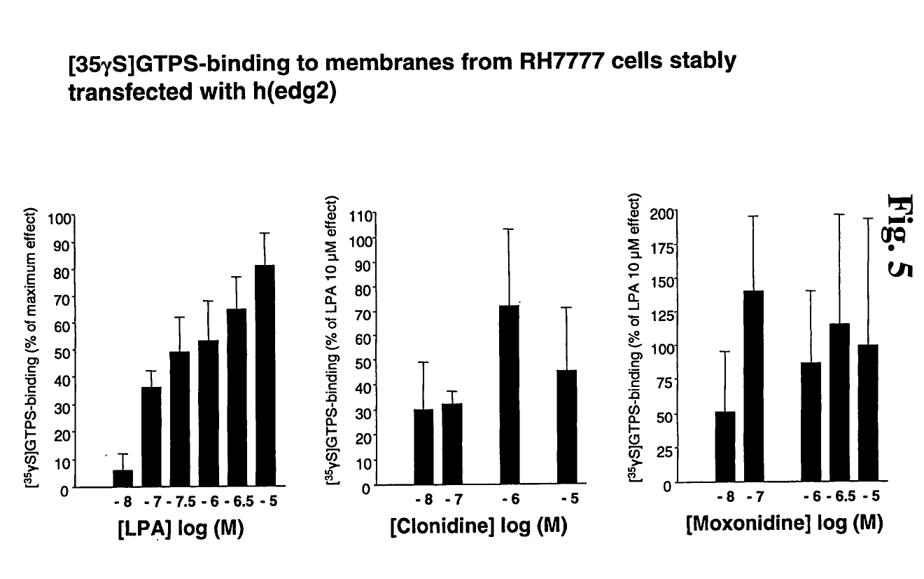

In the experiments with imidazoline and guanidine derivatives, 3 μM rauwolscine was present in the incubation buffer unless stated otherwise. This experimental condition was the same as in previous analogous experiments in the rat vena cava (Molderings and Göthert, 1998) and excluded the possibility that the compounds might mediate an effect via activation of 2′ adrenoceptors. Rauwolscine (3 μM) given alone did not significantly modify the veratridine-induced [³H]noradrenaline release (n=6; results not shown). In the presence of 3 μM rauwolscine (standard condition in the experiments with imidazolines, guanidines and cannabinoid ligands), BDF 6143 and agmatine (FIG. 1) as well as clonidine (FIG. 2A) concentration-dependently inhibited the veratridine-induced [3H]noradrenaline release. In addition, several further imidazolines and guanidines were studied at a standard concentration of 100 μM; not only BDF6143, agmatine (FIG. 1, Table 1) and clonidine (FIG. 2, Table 1) but also cirazoline, aganodine and DTG inhibited the veratridine-induced [³H]noradrenaline release (Table 1). Under the assumption that all active compounds produce the same maximum effect and exhibit approximately parallel concentration-response curves (which is supported by the similar shape of the concentration-response curves shown in FIGS. 1 and 2), the inhibition obtained by 100 μM of the respective compound may be considered to be a rough qualitative estimate of their potency. Under this assumption, the results are compatible with the following rank order of inhibitory potency: cirazoline clonidine aganodine>DTG BDF6143 agmatine. In contrast, the catecholamine noradrenaline and the imidazoline moxonidine (the latter up to a concentration of 1000 μM) failed to modify the veratridine-induced [³H]noradrenaline release (FIG. 1, Table 1).

In the absence of rauwolscine, clonidine also concentration-dependently inhibited veratridine-evoked [³H]noradrenaline release (FIG. 2A, open circles). The concentration-response curve of clonidine in the presence of 3 μM rauwolscine did not substantially differ from that in the absence of rauwolscine (FIG. 2A, closed circles). However, this curve was shifted to the right by 30 μM rauwolscine (FIG. 2A, closed triangles) and SR141716A (FIG. 2A; 10 μM, closed squares); in the presence of this SR141716A concentration, 10 μM clonidine even acted facilitatory on [³H]noradrenaline release (FIG. 2A). SR141716A and LY320135 at concentrations of 1 and 10 μM, respectively, counteracted the inhibition produced by 100 μM clonidine (FIG. 2B). SR141716A and LY 320135 given alone did not affect the veratridine-evoked [³H]noradrenaline release (result not shown). When the PC12 cells were pretreated with 100 μM clonidine for 72 h, 100 μM clonidine present from the onset of incubation with HEPES buffer until the end of the experiment did not inhibit but increased evoked [³H]noradrenaline release (171±24% of veratridine-evoked (3H]noradrenaline release in the respective control experiments; n=6, P<0.05).

Effects of Cannabinoid Receptor Agonists and Interaction with Antagonists

In the presence of 3 μM rauwolscine, WIN55,212-2 concentration-dependently inhibited evoked [³H]noradrenaline release, whereas 100 μM WIN55,212-3 did not (FIG. 3). CP55,940 at a concentration of 0.3 μM also significantly inhibited evoked tritium overflow, whereas at 1 μM no inhibition was found (FIG. 3) and at 10 μM even a facilitation was induced (FIG. 3, open columns).

The inhibitory effect of 0.3 μM CP55,940 was antagonized by 1 μM SR141716A (FIG. 3, solid column). Anandamide at a concentration of 1 μM did not affect, whereas 100 μM andandamide enhanced veratridine-evoked [³H]noradrenaline release (FIG. 3).

Effects of Lysophospholipid Receptor Agonists and Interaction with Antagonists

In the absence of interacting drugs, 1-oleoyl-LPA concentration-dependently inhibited the evoked [3H]noradrenaline release (FIG. 4A). In contrast, 10 μM S1P was ineffective (101.7%±31.3% of the veratridine-evoked [³H]noradrenaline release in the respective control experiments; n=6). The concentration-response curve for 1-palmitoyl-LPA was bell-shaped (FIG. 4A); evoked [³H]noradrenaline release was concentration-dependently increased by up to 1 μM 1-palmitoyl-LPA, whereas 10 μM did not alter evoked [³H]noradrenaline release. The inhibitory effect of 10 μM 1-oleoyl-LPA was counteracted by 30 μM rauwolscine and 1 μM SR141716A (FIG. 4B); 10 μM LY320135 even reversed the inhibitory effect of 10 μM 1-oleoyl-LPA to a facilitation (198.8%±39.9% of the evoked [³H]noradrenaline release in the respective control experiments, n=6; P<0.05 compared with the respective control experiments). When the PC12 cells were pretreated with 100 μM clonidine for 72 h, 10 μM 1-oleoyl-LPA did not inhibit but tended to increase evoked ³H]noradrenaline release (FIG. 4B).

Radioligand Binding Experiments

In the radioligand binding experiments, 16.8 nM (3H]rauwolscine failed to label specific 2-adrenoceptor binding sites as defined by addition of unlabelled rauwolscine (10 μM). [³H]Rauwolscine binding amounted to 134.0±7.1 (n=4) and 132.8±7.0 fmol/mg protein (n=4) in the absence and presence of 10 μM rauwolscine, respectively (corresponding to 0.19±0.01% and 0.19±0.01% of the radioactivity added to each assay, respectively). Analogously, [³H]SR141716A 10 nM did not label specific cannabinoid CB₁ binding sites as defined by addition of CP55,940 (3 μM). In the absence and presence of 3 μM CP55,940, binding of [³H]SR141716A amounted to 84.1±4.8 (n=4) and 85.2±4.1 fmol/mg protein (n=4) corresponding to 4.64±0.13% and 4.71±0.12% of the radioactivity added to each assay, respectively.

Evidence for Receptor Expression by PCR Experiments

Primer pairs suitable to identify the cDNAs for the rat_{2A} and CB₁ cannabinoid receptors were applied in PCR experiments. In control experiments fragments were amplified by PCR from the genomic DNA from rat blood with the same primer pairs. Sequencing of these fragments revealed that they code for the r_{2A}-adrenoceptor and the rCB₁ receptor. In contrast, no message for the r_{2A} and the rCB₁ receptors were found with the respective primer pairs in the cDNA from the undifferentiated PC12 cells (results not shown).

Genomic DNA prepared from rat blood was also used to prove whether the chosen primer pairs for lysophospholipid receptors were suitable to identify the corresponding edg messages. After PCR amplification of this genomic DNA, PCR products were found that were identified by sequencing as partial sequences of edg1, edg2, edg3, edg4, edg5, edg6, edg7 and edg8 (results not shown). PCR amplification of PC12 cell cDNA using the same primer pairs resulted in PCR products coding for partial sequences of edg2, edg3, edg5 and edg7 but not of edg1, edg4, edg6 and edg8 (results not shown).

TABLE 1
Influence of imidazoline derivatives (cirazoline, clonidine,
BDF 6143, moxonidine), guanidine derivatives (aganodine,
DTG, agmatine) and noradrenaline at a concentration of
100 μM on [³H]noradrenaline release from PC12 cells.

| | Veratridine (1 mM)-induced |
|---|---|
| Drug | [³H]noradrenaline release |
| (100 μM) | (% of controls without drug) |
| Cirazoline | 17.4 ± 7.5% *** |
| Clonidine | 25.6 ± 12.2% * |
| Aganodine | 32.3 ± 5.7% ** |
| DTG | 63.3 ± 7.0% * |
| BDF 6143 | 74.7 ± 11.1% * |
| Agmatine | 76.4 ± 9.5% |
| Moxonidine | 81.6 ± 5.2% |
| Noradrenaline | 93.0 ± 11.5% |
| The cells were preincubated with culture medium containing 10 nM [³H]noradrenaline and, after 60-min exposure to [³H]noradrenaline-free culture medium, incubated with [³H]noradrenaline-free HEPES-buffered salt solution containing 15 mM KCl, 1 μM desipramine and 3 μM rauwolscine throughtout. Unlabelled noradrenaline or the imidazoline or guanidine derivatives under study was | |
| # present from the onset of incubation with the HEPES buffer until the end of the experiment; 5-min stimulation with 1 mM veratridine was carried out 60 min after onset of incubation with the buffer. Evoked [³H]noradrenaline release was expressed as percentage of veratridine-evoked release in control experiments carried out in parallel without the drug under study. | |

Means ± SEM of 6-11 experiments in each group.
* P < 0.05,
** P < 0.01,
*** P < 0.001 (compared with the corresponding controls)

3. Agonist-Induced [³⁵S]GTPγS Binding

RH7777 cells (rat hepatoma cells) stably transfected with cDNA encoding the h(edg2) receptor and PC12 cells were homogenized in ice-cold 20 mM HEPES buffer containing 10 n mM EDTA (pH 7.4 at RT) with an ultraturrax homgenizer and centrifuged at 48,000 g, 4° C. for 15 min. The pellet was resuspended in ice-cold 20 mM HEPES buffer containing 0.1 mM EDTA (pH 7.4 at RT) and recentrifuged at 48,000 g, 4° C. for 15 min. The final pellet was resuspended in 20 mM HEPES buffer (pH 7.4 at RT).

50 μg Membranes were incubated in 0.5 ml of GTP-binding buffer containing GDP 0.1 μM, rauwolscine 10 μM (in the experiments with membranes from RH7777 cells), [³⁵S]GTPγs 0.05 nM and indicated test compound (LPA, clonidine or moxonidine diluted in assay buffer containing 1% fatty acid free BSA) for 45 min at 30° C. The incubation was terminated by filtering over GF/B filters with a cell harvester, and the filters were washed once with 5 ml of ice-cold assay buffer. Radioactivity was counted by liquid scintillation spectrometry at an efficiency of >95%.

The results from this experiment confirming the conclusions according to the present invention are shown in the FIG. 5 (FIG. 5A=LPA; FIG. 5B=clonidine; FIG. 5C=moxonidine):

[³⁵S]GTPγS-binding to membranes from RH7777 cells stably transfected with h(edg2) receptors in response to the endogenous edg2 receptor ligand lysophospatidic acid (LPA) and the imidazoline compounds clonidine and moxonidine. Data are expressed as mean values+SEM of 3-7 experiments in each series. Maximum stimulation of [³⁵S]GTPγS amounted to 500-1500 dpm on average.

From the results and the data of the [³⁵S]GTPγS-binding shown in FIG. 5 it is clearly evident that clonidine und moxonidine, like the endogenous ligand for edg2-receptors (LPA, lysophospatidic acid), provoke an increase of the [³⁵]GTPγS-binding in nanomolar concentrations, and this applies to the same extent as for the endogenous ligand. This means that both compounds seem to be full agonists of the edg2-receptor. The observation that moxonidine, besides clonidine, also acts as a potent agonist allows the conclusion that the edg2-receptor is identical to the I₁-imidazoline receptor, because for a potentially alternative imidazoline receptor which could have been deemed to be accurate moxonidine showed no effect. Thus, in line with earlier observations from earlier publications, the results of the present experiments lead to the conclusion that the I₁-imidazoline receptor involved in the centrally mediated lowering of the blood pressure is identical to the edg2-receptor. These conclusions are also in line with the distribution of edg2-receptors in the human organism.

### REFERENCES

Bandoh K., Aoki J., Hosono H., Kobayashi S., Kobayashi T., Murakami-Murofushi K., Tsujimoto M., Arai H., Inoue K., 1999. Molecular cloning and characterization of a novel human G-protein-coupled receptor, EDG7, for lysophosphatidic acid. J. Biol. Chem. 274, 27776-27785.
Bisogno T., Katayama K., Melck D., Ueda N., De Petrocellis L., Yamamoto S., Di Marzo V., 1998. Biosynthesis and degradation of bioactive fatty acid amides in human breast cancer and rat pheochromocytoma cells—implication for cell proliferation and differentiation. Eur. J. Biochem. 15,634-642.
Bönisch H., Boer R., Dobler M., Schudt C., 1990. Pharmacological characterization of muscarinic receptors of PC12 (rat phaeochromocytoma) cells. Naunyn-Schmiedeberg's Arch. Pharmacol. 341, 158-164.
Duzic E., Lanier S. M., 1992. Factors determing the specifity of signal transduction by guanine nucleotide-binding protein-coupled receptors. J. Biol. Chem. 267, 24045-24052.
Felder C. C., Joyce K. E., Briley E. M., Glass M., Mackie K. P., Fahey K. J., Cullinan G. J., Hunden D. C., Johnson D. W., Chaney M. O., Koppel G. A., Brownstein M., 1998. LY320135, a novel cannabinoid CB₁ receptor antagonist, unmasks coupling of the CB₁ receptor to stimulation of cAMP accumulation. J. Pharmacol. Exp. Ther. 284, 291-297.
Friedrich U., Bönisch H., 1986. The neuronal noradrenaline transport system of PC12 cells: kinetic analysis of the interaction between noradrenaline, Na⁺ and Cl− in transport. Naunyn-Schmiedeberg's Arch. Pharmacol. 333, 246-252.
Fuder H., Schwarz P., 1993. Desensitization of inhibitory prejunctional ₂-adrenoceptors and putative imidazoline receptors on rabbit heart sympathic nerves. Naunyn-Schmiedeberg's Arch. Pharmacol. 348, 127-133.
Gatti G., Madeddu L., Pandiella A., Pozzan T., Meldolesi J., 1988. Second-messenger generation in PC12 cells. Interactions between cyclic AMP and Ca²⁺ signals. Biochem. J. 255, 753-760.
Göthert M., Molderings G. J., 1991. Involvement of presynaptic imidazoline receptors in the 2-adrenoceptor-independent inhibition of noradrenaline release by imidazoline derivatives. Naunyn-Schmiedeberg's Arch. Pharmacol. 343, 271-282.
Gollasch M., Hescheler J., Spicher K., Klinz F. J., Schultz G., Rosenthal W., 1992. Inhibition of Ca²⁺ channels via ₂-adrenergic and muscarinergic receptors in pheochromocytoma (PC12) cells. Am. J. Physiol. 260, C1282-C1289.
Greene L. A., Tischler A. S., 1976. Establishment of a noradrenergic clonal line of rat adrenal pheochromocytoma cells which respond to nerve growth factor. Proc. Natl. Acad. Sci. USA 73, 2424-2428.
Harder R., Bönisch H., 1984. Large-scale preparation of plasma membrane vesicles from PC12 pheochromocytoma cells and their use in noradrenaline transport studies. Biochim. Biophys. Acta 775, 95-104.
Im D. S., Heise C. E., Harding M. A., George S. R., O'Dowd B. F., Theodorescu D., Lynch K. R., 2000. Molecular cloning and characterization of a lysophosphatidic acid receptor, EDG-7, expressed in prostate. Mol. Pharmacol. 57: 753-759.
Kim W. K., Hawthorn M. H., Rabin R. A., 1993. Chronic ethanol exposure potentiates muscarinic receptor and ₂-adrenoceptor-mediated inhibition of cAMP accumulation in PC12 cells. Eur. J. Pharmacol. 247, 89-92.
Kiss Z., 1999. Anandamide stimulates phospholipase D activity in PC12 cells but not in NIH 3T3 fibroblasts. FEBS Letters 447, 209-212.
Molderings G. J., Göthert M., 1998. Presynaptic imidazoline receptors mediate inhibition of noradrenaline release from sympathetic nerves in rat blood vessels. Fund. Clin. Pharmacol. 12, 388-397.
Molderings G. J., Göthert M., 1999. Imidazoline binding sites and receptors in cardiovascular tissue. Gen. Pharmacol. 32, 17-22.
Molderings G. J., Likungu J., Göthert M., 1999. Presynaptic cannabinoid and imidazoline receptors in the human heart and their potential relationship. Naunyn-Schmiedeberg's Arch. Pharmacol. 360,157-164.
Reed J. K., England D., 1986. The effect of nerve growth factor on the development of sodium channels in PC12 cells. Biochem. Cell Biol. 64, 1153-1159.
Rinaldi-Carmona M., Barth F., Heaulme M., Alonso R., Shire D., Congy C., Soubrie P., Breliere J. C., Le Fur G., 1995. Biochemical and pharmacological characterization of SR141716A, the first potent and selective brain cannabinoid receptor antagonist. Life Sci. 56, 1941-1947.
Rudy B., Kirschenbaum B., Rukenstein A., Greene L. A. (1987) Nerve growth factor increases the number of functional Na channels and induces TTX-resistant Na channels in PC12 pheochromocytoma cells. J. Neurosci. 7, 1613-1625.
Schwegler J. S., Bönisch H., 1986. Receptor binding with a neuronal cell line (PC-12). Naunyn-Schmiedeberg's Arch. Pharmacol. 332 (Suppl.), R78.
Starke K., 1981. Presynaptic receptors. Annu. Rev. Pharmacol. Toxicol. 21, 7-30.
Tahir S. K., Trogadis J. E., Stevens J. K., Zimmerman A. M., 1992. Cytoskeletal organization following cannabinoid treatment in undifferentiated and differentiated PC12 cells. Biochem. Cell Biol. 70, 1159-1173.
Trendelenburg A. U., Sutej I., Wahl C. A., Molderings G. J., Rump L. C., Starke K., 1997. A re-investigation of questionable subclassification of presynaptic ₂-autoreceptors: rat vena cava, rat atria, human kidney and guinea-pig urethra. Naunyn-Schmiedeberg's Arch. Pharmacol. 356, 721-737.
Ulbricht W., 1998. Effects of veratridine on sodium currents and fluxes. Rev. Physiol. Biochem. Pharmacol. 133, 1-54.

All publications, including but not limited to patents and patent applications, cited in this specification are herein incorporated by reference as if each individual publication were specifically and individually indicated to be incorporated by reference herein as though fully set forth.

1-15. (canceled)

16. A method of treating or inhibiting hypertension in a mammal in need thereof, said method comprising administering to said mammal a therapeutically effective amount of an edg-receptor agonist or a pharmaceutically acceptable salt thereof.

17. A method according to claim 16, wherein the edg-receptor agonist is a highly selective I1-receptor agonist, essentially devoid of alpha-2-receptor agonist activity.

18. A method according to claim 17, wherein the edg-receptor agonist has an imidazoline compound structure.

19. A method for determining whether a substance is a ligand of an edg-encoded lysohospholipid receptor, said method comprising:
a) contacting
cells expressing the edg-encoded lysohospholipid receptor polypeptide, or
a receptor membrane preparation comprising the edg-encoded lysohospholipid receptor polypeptide,
with a labeled ligand in the presence and in the absence of said substance; and
b) measuring the binding of the labeled ligand to the edg-encoded lysohospholipid receptor.

20. A method according to claim 19, wherein said edg-encoded lysohospholipid receptor is a mammalian edg-encoded lysohospholipid receptor.

21. A method according to claim 19, for determining whether a test substance is a ligand of an edg-encoded lysohospholipid receptor, wherein said method comprises [35S] GTPγS-binding to a membrane preparation comprising said edg-encoded lysohospholipid receptor polypeptide.

22. A method according to claim 21, wherein said edg-encoded lysohospholipid receptor is a mammalian edg-encoded lysohospholipid receptor, and the membrane preparation is prepared from RH7777 cells comprising said edg-encoded lysohospholipid receptor polypeptide.

23. A method for determining whether a substance modulates the interaction of a ligand with an edg-encoded lysohospholipid receptor polypeptide, said method comprising:
contacting cells, which express on the surface thereof an edg-encoded lysohospholipid receptor polypeptide, with a test substance to be screened in the presence of a ligand which binds to the receptor and under conditions permitting binding of the ligand to the receptor, wherein the receptor is associated with a second component capable of providing a detectable signal in response to binding of the ligand to the receptor; and
determining whether the test substance modulates the interaction between said ligand and the edg-encoded lysohospholipid receptor polypeptide by detecting an increase or decrease in the signal generated by binding of the ligand with the receptor.

24. A method according to claim 23, wherein said edg-encoded lysohospholipid receptor polypeptide is a mammalian edg-encoded lysohospholipid receptor polypeptide.

25. A method according to claim 23, wherein the test substance is effective to treat or inhibit a disfunction, disorder or disease selected from the group consisting of dysfunctions, disorders or diseases of the cardiovascular system, of the nervous system, of glucose and insulin metabolism and involving increased sympathetic tonicity.

26. A method according to claim 25, wherein the test substance is effective to treat or inhibit a cardiovascular or heart condition selected from the group consisting of blood pressure disorders, myocardial ischaemia, and ischaemic preconditioning, or to exert a cardioprotective activity.

27. A method according to claim 26, wherein the test substance is effective to treat hypertension or vasodilatation.

28. A method according to claim 23, wherein said cells are isolated rat pheochromocytoma PC12 cells comprising an edg-encoded lysohospholipid receptor.

29. A method according to claim 28, wherein the edg-encoded receptor comprised by said isolated rat pheochromocytoma PC12 cells is a nordrenaline release-inhibiting receptor.

30. A method according to claim 28, wherein the edg-encoded lysohospholipid receptor is encoded by a gene selected from the group consisting of edg2, edg3, edg5 and edg7.

31. A method according to claim 28, wherein the isolated rat pheochromocytoma PC12 cells are devoid of alpha-2-receptors or CB1-receptors or both.

32. A method for determining whether a substance inhibits or antagonizes interaction of a ligand with an edg-encoded lysohospholipid receptor polypeptide, said method comprising:
a) contacting cells, which express on the surface thereof an edg-encoded lysohospholipid receptor polypeptide, with a substance to be screened in the presence of a ligand which binds to the receptor and under conditions which permit ligand binding to the receptor, wherein the receptor is associated with a second component capable of providing a detectable signal in response to the binding of the ligand to the receptor; and
b) determining whether the substance inhibits or antagonizes the interaction of the ligand and the edg-encoded lysohospholipid receptor polypeptide by detecting a decrease in the signal generated by binding of the ligand with the receptor.

33. A method according to claim 32, wherein said edg-encoded lysohospholipid receptor polypeptide is a mammalian edg-encoded lysohospholipid receptor polypeptide.

34. A method according to claim 32, wherein the test substance is effective to treat or inhibit a disfunction, disorder or disease selected from the group consisting of dysfunctions, disorders or diseases of the cardiovascular system, of the nervous system, of glucose and insulin metabolism and involving increased sympathetic tonicity.

35. A method according to claim 34, wherein the test substance is effective to treat or inhibit a cardiovascular or heart condition selected from the group consisting of blood pressure disorders, myocardial ischaemia, and ischaemic preconditioning, or to exert a cardioprotective activity.

36. A method according to claim 35, wherein the test substance is effective to treat hypertension or vasodilatation.

37. A method according to claim 32, wherein said cells are isolated rat pheochromocytoma PC12 cells comprising an edg-encoded lysohospholipid receptor.

38. A method according to claim 37, wherein the edg-encoded receptor comprised by said isolated rat pheochromocytoma PC12 cells is a nordrenaline release-inhibiting receptor.

39. A method according to claim 37, wherein the edg-encoded lysohospholipid receptor is encoded by a gene selected from the group consisting of edg2, edg3, edg5 and edg7.

40. A method according to claim 37, wherein the isolated rat pheochromocytoma PC12 cells are devoid of alpha-2-receptors or CB1-receptors or both.

41. A method for determining whether a substance is an agonist to an edg-encoded lysohospholipid receptor polypeptide, said method comprising:
a). contacting cells, which express on the surface thereof an edg-encoded lysohospholipid receptor polypeptide, with a test substance, wherein the receptor is associated with a second component capable of providing a detectable signal in response to activation of the receptor; and
b) determining whether contact with the test substance effects a signal generated by activation of the edg-encoded lysohospholipid receptor polypeptide;
wherein a ligand which binds to the receptor and is distinct from said test substance is used as a positive control.

42. A method according to claim 41, wherein said edg-encoded lysohospholipid receptor polypeptide is a mammalian edg-encoded lysohospholipid receptor polypeptide.

43. A method according to claim 41, wherein the test substance is effective to treat or inhibit a disfunction, disorder or disease selected from the group consisting of dysfunctions, disorders or diseases of the cardiovascular system, of the nervous system, of glucose and insulin metabolism and involving increased sympathetic tonicity.

44. A method according to claim 43, wherein the test substance is effective to treat or inhibit a cardiovascular or heart condition selected from the group consisting of blood pressure disorders, myocardial ischaemia, and ischaemic preconditioning, or to exert a cardioprotective activity.

45. A method according to claim 44, wherein the test substance is effective to treat hypertension or vasodilatation.

46. A method according to claim 41, wherein said cells are isolated rat pheochromocytoma PC12 cells comprising an edg-encoded lysohospholipid receptor.

47. A method according to claim 46, wherein the edg-encoded receptor comprised by said isolated rat pheochromocytoma PC12 cells is a nordrenaline release-inhibiting receptor.

48. A method according to claim 46, wherein the edg-encoded lysohospholipid receptor is encoded by a gene selected from the group consisting of edg2, edg3, edg5 and edg7.

49. A method according to claim 46, wherein the isolated rat pheochromocytoma PC12 cells are devoid of alpha-2-receptors or CB1-receptors or both.

50. A method for determining whether a test substance is a modulator, an agonist or antagonist to a noradrenaline release-inhibiting edg-encoded lysohospholipid receptor polypeptide, said method comprising:
a) loading cells, which express on the surface thereof an edg-encoded lysohospholipid receptor polypeptide, with labelled noradrenaline;
b) contacting the labelled noradrenaline loaded cells with a test substance;
c) adding veratridine to the labelled noradrenaline loaded cells in order to evoke noradrenaline-release in the presence of the test substance, and
d) determining whether the interaction of the test substance with the edg-encoded lysohospholipid receptor polypeptide modulates, agonizes or antagonizes release of noradrenaline.

51. A method according to claim 50, wherein said edg-encoded lysohospholipid receptor polypeptide is a mammalian edg-encoded lysohospholipid receptor polypeptide.

52. A method according to claim 50, wherein the test substance is effective to treat or inhibit a disfunction, disorder or disease selected from the group consisting of dysfunctions, disorders or diseases of the cardiovascular system, of the nervous system, of glucose and insulin metabolism and involving increased sympathetic tonicity.

53. A method according to claim 52, wherein the test substance is effective to treat or inhibit a cardiovascular or heart condition selected from the group consisting of blood pressure disorders, myocardial ischaemia, and ischaemic preconditioning, or to exert a cardioprotective activity.

54. A method according to claim 53, wherein the test substance is effective to treat hypertension or vasodilatation.

55. A method according to claim 50, wherein said cells are isolated rat pheochromocytoma PC12 cells comprising an edg-encoded lysohospholipid receptor.

56. A method according to claim 55, wherein the edg-encoded receptor comprised by said isolated rat pheochromocytoma PC12 cells is a nordrenaline release-inhibiting receptor.

57. A method according to claim 55, wherein the edg-encoded lysohospholipid receptor is encoded by a gene selected from the group consisting of edg2, edg3, edg5 and edg7.

58. A method according to claim 55, wherein the isolated rat pheochromocytoma PC12 cells are devoid of alpha-2-receptors or CB1-receptors or both.
